(19) United States
(12) Patent Application Publication
(10) Pub. No.: US 2005/0192340 A1
(43) Pub. Date: Sep. 1, 2005

(54) Simvastatin formulations and methods of making same

(76) Inventors: Moshe Flashner-Barak, Petach Tikva (IL); E. Itzhak Lerner, Petach Tikva (IL); Vered Rosenberger, Jerusalem (IL); Naomi Moldavski, Jerusalem (IL)

Correspondence Address: KENYON & KENYON, ONE BROADWAY, NEW YORK, NY, 10004, US

(21) Appl. No.: 10/981,953

(22) Filed: Nov. 5, 2004

Related U.S. Application Data
(60) Provisional application No. 60/517,650, filed on Nov. 5, 2003.

(51) Int. Cl. A61K 31/401; A61K 31/366; A61K 31/225

(52) U.S. Cl. 514/423; 514/460; 514/548

(57) ABSTRACT

The invention encompasses a compositions of at least one statin, at least one pharmaceutically acceptable solvent, and at least one surface active agent. In the composition, about 9% to about 50% by weight of the statin is hydrolyzed from a closed lactone form to an open hydroxy acid form when the composition is placed in an aqueous acidic solution. The invention also encompasses method of making the composition and methods of treating high cholesterol, multiple sclerosis, and/or Alzheimer's disease using the compositions described herein.

### RELATED APPLICATIONS

This application claims the benefit of U.S. Provisional Application No. 60/517,650, filed on Nov. 5, 2003.

### FIELD OF THE INVENTION

The present invention is directed to statin compositions and to methods of preparing the compositions; preferably, the statin is simvastatin. Also, the present invention is directed to methods of treating high cholesterol, multiple sclerosis, and/or Aizheimer's disease by administering a therapeutically effective amount of statin using the compositions of the invention.

### BACKGROUND OF THE INVENTION

The statin class of drugs that lower cholesterol levels are among the most commercially successful drugs. In 1987 with the introduction of lovastatin, the statin drugs first became available and since then, there has been a constant effort to introduce new, improved anti-cholesterol compounds. Compounds such as pravastatin, fluvastatin, simvastatin, and atorvastatin, to name a few, have since been introduced to compete with lovastatin. The desire to design and introduce new better performing “superstatins” has continued unabated.

The statin drugs' mechanism of action has been elucidated in some detail. Statins apparently interfere with the synthesis of cholesterol and other sterols in the liver by competitively inhibiting the 3-hydroxy-3-methyl-glutaryl-coenzyme A reductase enzyme (“HMG-CoA reductase”). HMG-CoA reductase catalyzes the conversion HMG to mevalonate, which is the rate determining step in the biosynthesis of cholesterol, thus inhibition leads to a reduction in the concentration of cholesterol in the liver.

The statins, as a class of drugs, contain a functional moiety that can exist either as a hydroxyl acid in an open non-ring structure with a hydroxyl in the delta position, or as a lactone in a six membered ring closed lactone structure. Although, the hydroxyl acid and lactone forms are chemically interchangeable, the open hydroxy acid form is apparently the preferred bioactive form. It is believed that basic conditions will non reversibly hydrolyze the closed lactone form to the salt of the open hydroxy acid form. In contrast, acidic conditions will lead to the lactonization of the open hydroxy acid form. The closed lactone form of the statins is apparently biologically inactive, as the lactone does not seem to inhibit HMG-CoA reductase enzyme, the target of statin class of compounds. In contrast, the open hydroxy acid form is apparently biologically active. Several of the statin drugs are delivered to the gastrointestinal (GI) tract as hydroxyl acid salts, while others, such as simvastatin, are delivered as closed lactones which are enzymatically hydrolyzed in the body to the apparently active moiety (active metabolite).

Simvastatin, as a member of the statin family of drugs, is a anti-hypercholesterolemic agent. Simvastatin is a synthetic analog of lovastatin, wherein the 8-acyl moiety is 2,2-dimethylbutyryl, and is chemically designated as 2,2-dimethylbutanoic acid (4R,6R)-6-[2[1S,2S,6R,8S,8aR)-1,2,6,7,8,8a-hexahydro-2,6-dimethyl-1-[2-(tetrahydro-4-hydroxy-6-oxo-2H-pyran-2-yl)ethyl]-1-napthalenyl ester (CAS Registry No. 79902-63-9). Simvastatin is commercially available as ZOCOR® sold by Merck Co, Inc. (West Point, Pa.). The preparation of simvastatin was apparently originally described in U.S. Pat. No. 4,444,784; the process seemed to involve deacylation of lovastatin followed by subsequent acylation with the 2,2-dimethylbutyryl moiety. Simvastatin may apparently also be prepared by the alpha alkylation of the lovastatin ester moiety as described in U.S. Pat. Nos. 4,582,915 and 4,820,850.

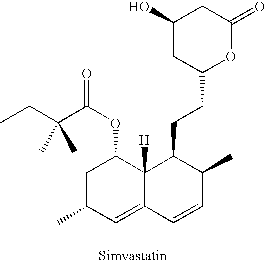

Simvastatin is apparently an effective drug, however, simvastatin's active metabolite bioavailability is only a few percent by weight. The low bioavailability is believed to be caused by competing metabolism of the simvastatin by cytochrome P enzymes in the gut wall and in the liver, the drug action site. Nevertheless, simvastatin is seen to be an efficacious drug in the treatment of elevated cholesterol levels.

Simvastatin is administered in the closed lactone form as a “pro-drug” of the active metabolite, the hydrolyzed hydroxyl acid form. As with other statins, simvastatin can exist either in a 3-hydroxy closed lactone form or an open hydroxy acid form. The closed lactone form is not believed to be an active inhibitor of HMG-CoA reductase, while the open hydroxy acid form is apparently biologically active and preferred. The condensation of the open hydroxy acid form to the closed lactone form occurs under acidic conditions (e.g., in the stomach where pH is about pH 4 or under). Consequently, it is desirable to prepare simvastatin in the open hydroxy acid form and limit the in vivo amount of inactive closed lactone form to avoid undesirable side effects. An illustration of the closed lactone form portion of a statin and the corresponding open hydroxy acid form is shown below.

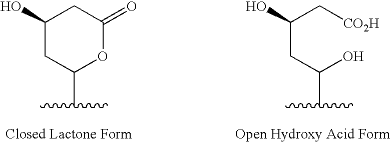

One method of improving the efficacy of simvastatin would be to find a way of delivering a preformed hydroxyl acid form of the drug to the body. Merck Co. Inc. has noted that delivering simvastatin to the stomach by conventional oral dosing in the lactone form leaves the drug essentially in the lactone form. See, WO 00/53173. As the acidic environment of the stomach favors the closed lactone form, the simvastatin lactone insolubility in the acidic aqueous environment of the stomach limits any hydrolysis of the lactone. Furthermore, the lack of hydrolytic enzymes and the stomach acidity level combine to favor the retention of the closed lactone form as simvastatin enters the small intestine, which is believed to be the main site of simvastatin absorption. Thus, the PCT Publication WO 00/53173 discusses the delivery of preformed simvastatin hydroxy acid salts with an enteric coated tablet so as to circumvent the acidic environment of the stomach and facilitate the absorption of the open hydroxy acid form in the neutral environment of the small intestine.

Despite improvements in statin delivery, conventional stomach delivery of simvastatin with increased bioavailability has yet to be achieved satisfactorily. Accordingly, the present invention addresses the prior art deficiencies and achieves higher bioavailability of the active statin form while not increasing the side effects observed with the closed lactone form.

### SUMMARY OF THE INVENTION

The invention encompasses compositions comprising a statin, at least one pharmaceutically acceptable solvent, and at least one surface active agent wherein about 9% to about 50% by weight of the statin may be hydrolyzed from a closed lactone form to an open hydroxy acid form when the composition is placed in an aqueous acidic solution. In one embodiment, the aqueous acidic solution may be 0.1 N HCl. Optionally, the composition may further comprise an effective amount of a pharmaceutically acceptable antioxidant. In another embodiment, the statin may be lovastatin, simvastatin, or a combination thereof; preferably, the statin is simvastatin. The statin may be present in an amount of about 1% to about 50% by weight of the composition.

In one embodiment of the composition, at least 90% by weight of the total amount of statin may be present in a closed lactone form statin. In another embodiment, the pharmaceutically acceptable solvent includes, but is not limited to, ethanol, propylene glycol, glycerol, isopropanol, butanol, or menthol; preferably, the pharmaceutically acceptable solvent is menthol. In another embodiment, the pharmaceutically acceptable solvent may be present in an amount of about 10% to about 75% by weight of the composition.

In one embodiment, the surface active agent includes, but is not limited to, glyceryl ester, polyoxyethylene glycol ester, polyoxyethylene glycol ether, polyoxyethylene sorbitan fatty acid ester, polyoxyethylene/polyoxypropylene copolymer, sodium lauryl sulfate, or sodium ducosate. The surface active agent may be present in an amount of about 5% to about 85% by weight of the composition. In another embodiment, the composition comprises at least two surface active agents wherein the first surface active agent may be polyoxyethylene sorbitan fatty acid ester or polyoxyethylene/polyoxypropylene copolymer and the second surface active agent may be sodium lauryl sulfate or sodium ducosate. Preferably, the first surface active agent is polyoxyethylene sorbitan fatty acid ester and the second surface active agent is sodium ducosate. In another embodiment, when the composition is dissolved in an aqueous acidic solution, at least 20% by weight of the dissolved statin may be hydrolyzed from a closed lactone form to an open hydroxy acid form. In yet another embodiment, the composition may be in a pharmaceutically acceptable dosage form.

One embodiment of the invention encompasses compositions comprising simvastatin; menthol; and polyoxyethylene sorbitan fatty acid ester, wherein about 9% to about 50% by weight of the simvastatin may be hydrolyzed from a closed lactone form to an open hydroxy acid form when the composition is placed in an aqueous acidic solution. Optionally, the composition may further comprise an effective amount of a pharmaceutically acceptable antioxidant. In one embodiment, the simvastatin may be present in an amount of about 1% to 50% by weight of the composition and menthol may be present in an amount of about 10% to about 75% by weight of the composition. In yet another embodiment, the composition comprises at least two surface active agents wherein the first surface active agent may be polyoxyethylene sorbitan fatty acid ester or polyoxyethylene/polyoxypropylene copolymer and the second surface active agent may be sodium lauryl sulfate or sodium ducosate. In one preferred embodiment, the polyoxyethylene sorbitan fatty acid ester may be present in an amount of 33% to about 57% by weight and the sodium lauryl sulfate or sodium ducosate may be present in an amount of about 5% to about 70% by weight. In one embodiment of the invention, when the composition is dissolved in an aqueous acidic solution, at least 20% by weight of the dissolved simvastatin may be hydrolyzed from a closed lactone form to an open hydroxy acid form.

The invention also encompasses methods of preparing simvastatin compositions comprising: heating menthol to a temperature of about 40° C. to about 60° C. to effect melting thereof; adding at least one first surface active agent to the menthol to form a first mixture; adding at least one second surface active agent containing a sulfate moiety and stirring until all components have dissolved to form a second mixture; adjusting the temperature of the second mixture to a temperature of about 50° C. to form a melt; adding simvastatin to the melt to form a simvastatin containing melt; and cooling the simvastatin containing melt to room temperature and dispensing the simvastatin containing melt into capsules. Alternatively, the melt may be dispensed into capsules and subsequently cooled to room temperature. Either method obtains capsules comprising a simvastatin melt at room temperature. In one embodiment, the method may further comprise adding at least one solid carrier to the cooled simvastatin containing melt to form a third mixture prior to dispensing the simvastatin containing melt into capsules. In another embodiment, the solid carrier may be at least one of microcrystalline cellulose, lactose, or sorbitol. One embodiment of the invention may further comprise cooling the third mixture to room temperature to form a powder.

The invention also encompasses methods of forming a solid simvastatin containing product comprising heating menthol to a temperature of about 40° C. to about 60° C. to effect the melting thereof; adding simvastatin to the melt; stirring the melt until all the simvastatin dissolves; adjusting the temperature of the melt to about 40° C.; and maintaining the temperature under stirring. Optionally, at least one pharmaceutically acceptable antioxidant may be added and dissolved or dispersed in the melt.

In one embodiment, at least one surface active agent is melted and/or dissolved in a pharmaceutically acceptable solvent which is later added to a menthol-simvastatin mixture. The method comprises dissolving or dispersing at least one surface active agent into a melt or solution; granulating the melt or solution with a solid carrier and at least one pharmaceutically acceptable solvent to form a solid granulate; drying the solid granulate; mixing the dry granulate and the menthol simvastatin melt; cooling the dry granulate and menthol simvastatin melt to room temperature with stirring; forming a powder of the mix; and using the powder to produce pharmaceutical dosage forms such as capsules, tablets, or sachets. Optionally, the method further comprises milling the dry granulate prior to mixing with the menthol-simvastatin melt. Optionally, at least one antioxidant may be added to the surface active agent.

One embodiment of the invention encompasses methods for treating a patient for elevated cholesterol levels, multiple sclerosis, or Alzheimer's disease comprising administering to a patient in need of such treatment a statin composition comprising a therapeutically effective amount of a statin, at least one pharmaceutically acceptable solvent, and at least one surface active agent, wherein about 9% to about 50% by weight of the statin may be hydrolyzed from a closed lactone form to an open hydroxy acid form when the composition is placed in an aqueous acidic solution. Optionally, the composition of the method may further comprise an effective amount of a pharmaceutically acceptable antioxidant. In one embodiment, the statin may be simvastatin present in an amount of about 1% to 50% by weight of the composition and the pharmaceutically acceptable solvent may be menthol present in an amount of about 10% to about 75% by weight of the composition.

In one embodiment of the method, the composition may comprise at least two surface active agents wherein the first surface active agent may be polyoxyethylene sorbitan fatty acid ester or polyoxyethylene/polyoxypropylene copolymer and the second surface active agent may be sodium lauryl sulfate or sodium ducosate. In a preferred embodiment, the polyoxyethylene sorbitan fatty acid ester may be present in an amount of 33% to about 57% by weight and the sodium lauryl sulfate or sodium ducosate may be present in an amount of about 5% to about 70% by weight. In another embodiment, when the composition is dissolved in 0.1N HCl, at least 30% by weight of the dissolved statin may be hydrolyzed from the closed lactone form to the open hydroxy acid form.

One embodiment of the invention encompasses methods for treating a patient for elevated cholesterol levels comprising administering to a patient in need of such treatment a statin composition comprising a therapeutically effective amount of a statin, menthol, and at least one surface active agent, wherein about 9% to about 50% by weight of the statin may be hydrolyzed from a closed lactone form to an open hydroxy acid form when the composition is placed in an aqueous acidic solution. Optionally, the composition of the method may further comprise an effective amount of a pharmaceutically acceptable antioxidant. In another embodiment, the statin may be simvastatin present in an amount of about 1% to 50% by weight of the composition and the menthol may be present in an amount of about 10% to about 75% by weight of the composition.

In one embodiment of the method, the composition may comprise at least two surface active agents wherein the first surface active agent may be polyoxyethylene sorbitan fatty acid ester or polyoxyethylene/polyoxypropylene copolymer and the second surface active agent may be sodium lauryl sulfate or sodium ducosate. In another embodiment of the method, the polyoxyethylene sorbitan fatty acid ester may be present in an amount of 33% to about 57% by weight and the sodium lauryl sulfate or sodium ducosate may be present in an amount of about 5% to about 70% by weight. In yet another embodiment, when the composition is dissolved in 0.1N HCl, at least 30% by weight of the dissolved statin may be hydrolyzed from the closed lactone form to the open hydroxy acid form.

One embodiment of the invention encompasses methods for treating a patient with multiple sclerosis comprising administering to a patient in need of such treatment a composition comprising a therapeutically effective amount of simvastatin dissolved in menthol, and at least one surface active agent, wherein about 9% to about 50% by weight of the simvastatin may be hydrolyzed from a closed lactone form to an open hydroxy acid form when the composition is placed in an aqueous acidic solution. Optionally, the composition of the method may further comprise an effective amount of a pharmaceutically acceptable antioxidant. In another embodiment, the statin may be simvastatin present in an amount of about 1% to 50% by weight of the composition and the menthol may be present in an amount of about 10% to about 75% by weight of the composition. In one embodiment of the method, the composition may comprise at least two surface active agents wherein the first surface active agent may be polyoxyethylene sorbitan fatty acid ester or polyoxyethylene/polyoxypropylene copolymer and the second surface active agent may be sodium lauryl sulfate or sodium ducosate.

In another embodiment of the method, the polyoxyethylene sorbitan fatty acid ester may be present in an amount of 33% to about 57% by weight and the sodium lauryl sulfate or sodium ducosate may be present in an amount of about 5% to about 70% by weight. In yet another embodiment, when the composition is dissolved in 0.1N HCl, at least 30% by weight of the dissolved statin may be hydrolyzed from the closed lactone form to the open hydroxy acid form.

One method of the invention encompasses methods for treating a patient with Alzheimer's disease comprising administering to a patient in need of such treatment a composition comprising a therapeutically effective amount of simvastatin dissolved in menthol, and at least one surface active agent, wherein about 9% to about 50% by weight of the simvastatin may be hydrolyzed from a closed lactone form to an open hydroxy acid form when the composition is placed in an aqueous acidic solution. Optionally, the composition of the method may further comprise an effective amount of a pharmaceutically acceptable antioxidant. In one embodiment, the surface active agent may be polyoxyethylene sorbitan fatty acid ester, polyoxyethylene/polyoxypropylene copolymer, sodium lauryl sulfate, or sodium ducosate.

### DETAILED DESCRIPTION OF THE INVENTION

The present invention encompasses statin formulations, which are delivered conventionally to the stomach and have a significantly higher bioavailability of the active metabolite, i.e. the open hydroxy acid form statin. Without being limited by theory, it is believed that the main reason for the low bioavailability of statins is that statins are unchanged when delivered to the stomach, i.e. the closed lactone form of the statin is insoluble in the acidic environment of the stomach and does not hydrolyze to the biologically active open hydroxy acid form. Accordingly, statin activity may be enhanced by partial hydrolysis of the dissolved closed lactone form statin to the open hydroxy acid form statin. The present invention is based in part on the judicial selection of pharmaceutically acceptable solvent(s) and surface active agent(s) to increase solubilization and hydrolysis of the closed lactone form statin, thus, converting the biologically inactive closed lactone form statin to the biologically active open hydroxy acid form statin in the stomach, thereby increasing the amount of available biologically active statin without increasing the overall amount of statin dosed.

Bioavailability pharmacokinetic studies of the compositions of the invention demonstrated that the bioavailability of the active metabolite, i.e. the open hydroxy acid form, was at least five times available as compared to conventional simvastatin formulations (closed lactone form). The discovery effectively places the compositions of the present invention into a “superstatin” category. It was also discovered that the relative concentration of the closed lactone form statin was unchanged indicating that the compositions of the invention will not enhance the side effects commonly associated with the closed lactone form statin. The invention also encompasses methods for making the compositions and methods of treating high cholesterol levels, multiple sclerosis, and/or Alzheimer's disease using the compositions of the invention.

The invention encompasses compositions comprising at least one statin, at least one pharmaceutically acceptable solvent, and at least one surface active agent. Optionally, the composition may include at least one antioxidant. In one embodiment, the statin of the composition may be in the closed lactone form, and as the statin solubilizes a portion of the dissolved closed lactone form is hydrolyzed to the open hydroxy acid form when the composition is partially or completely dissolved in acid. Typically, about 9% to about 50% by weight of the simvastatin may be hydrolyzed from a closed lactone form to an open hydroxy acid form when the composition is placed in an aqueous acidic solution. In one embodiment, about 15% by weight to about 50% by weight of the statin, and preferably, from about 20% to about 40% by weight of the statin may be hydrolyzed from a closed lactone form to an open hydroxy acid form when the composition is placed in an aqueous acidic solution. The aqueous acidic solution may be 0.1 N HCl.

The statins used in the invention may be obtained either commercially or from methods commonly known in the art. Preferably, the statin is in a pharmaceutically acceptable form. As used herein, the term “pharmaceutically acceptable” means approved by a regulatory agency of the Federal or a state government or listed in the U.S. Pharmacopeia or other generally recognized pharmacopeia for use in animals, and more particularly in humans. The statin may be present either in the closed lactone form, in the open hydroxy acid form, or a combination thereof. When present as a combination of the closed lactone form and the open hydroxy acid form, preferably the statin may be at least 90% by weight in the closed lactone form, more preferably at least 95% by weight, and most preferably the statin is present in the closed lactone form in at least 98% by weight of the total amount of statin prior to hydrolysis. Statin drugs include, but are not limited to, lovastatin, simvastatin, esters thereof, or lactone forms thereof. Preferably, the statin is simvastatin.

The statin may be present from about 1% to about 50% by weight of the composition, and preferably, in about 8% by weight of the composition. Alternatively, the statin may be present in an amount of about 1 mg to about 80 mg per dose unit, more preferably, the statin may be present in an amount of about 5 mg to about 40 mg per dose unit, and most preferably, the statin may be present in an amount of about 20 mg per dose unit.

Typically, the pharmaceutically acceptable solvent includes, but is not limited to, at least one of ethanol, propylene glycol, glycerol, isopropanol, butanol, or menthol. Preferably, the pharmaceutically acceptable solvents include, but are not limited to, at least one of menthol or ethanol. More preferably, the pharmaceutically acceptable solvent is menthol. Typically, the pharmaceutically acceptable solvent may be present in an amount sufficient to partially dissolve the statin. The pharmaceutically acceptable solvent may be present in an amount of about 10% to about 75% by weight of the composition, and preferably, present in an amount of about 41% by weight of the composition. Alternatively, the pharmaceutically acceptable solvent may be present in about 10 mg to about 250 mg per dose unit, and more preferably in about 25 mg to about 100 mg per dose unit.

The surface active agents of the invention include emulsifying agents such as those commonly known to one skilled in that art. See, REMINGTON THE SCIENCE AND PRACTICE OF PHARMACY (A. Gennaro, ed. 20^{th} Ed. 2000). Preferably, the surface active agents include emulsifying agents such as non-ionic undissociated surfactants which possess hydrophilic and lipophilic groups within the molecule and/or sulfate containing surfactants. Surface active agents include, but are not limited to, glyceryl esters, polyoxyethylene glycol esters, polyoxyethylene glycol ethers, polyoxyethylene sorbitan fatty acid esters, sulfate containing surfactants, or polyoxyethylene/polyoxypropylene copolymers. Preferably, the surface active agents include polyoxyethylene sorbitan fatty. acid esters, polyoxyethylene/polyoxypropylene copolymers known as poloxamers, sodium lauryl sulfate, or sodium ducosate. For example, commercially available surface active agents include TWEEN® 80, TWEEN® 20 (e.g. Polysorbitan 80, Polysorbitan 20), or Poloxamer 407. In one embodiment, the surface active agent is present in an effective amount to hydrolyze from about 15% by weight to about 50% by weight of the statin, and preferably, the surface active agent is present in an effective amount to hydrolyze from about 20% to about 40% by weight of the statin. When a surface active agent is present in the composition, the statin can achieve partial or full solubilization of the closed lactone form in a stimulated gastric environment, e.g. 0.1 N HCl. The surface active agent should be present in sufficient amount such that about at least 20% by weight of the total amount of dissolved statin is hydrolyzed to the open hydroxy acid form of the statin. Preferably, the surface active agent is present in a sufficient amount such that about at least 30% by weight of the total amount of dissolved statin is hydrolyzed to the open hydroxy acid form, and more preferably, at least about 40% by weight of the total amount of dissolved statin.

The surface active agent may be present in an amount of about 5% to about 85% by weight of the composition, preferably from about 40% to about 80% by weight. Alternatively, individual surface active agents may be present in an amount of about 5% by weight of the composition to about 75% by weight, and preferably, from about 9% to about 70% by weight. In one embodiment, when the surface active agent is sodium ducosate, the surface active agent may be present in an amount of about 10% to about 75% by weight of the composition, and preferably in an amount of about 17% to about 35% by weight of the composition. Alternatively, when the surface active agent is sodium ducosate, the surface active agent may be present in an amount of about 20 mg to about 185 mg per dose unit, preferably, in an amount of about 42 mg per dose unit. In another embodiment, when the surface active agent is a polyoxyethylene sorbitan fatty acid ester, the surface active agent may be present in an amount of about 20% to about 75% by weight of the composition, preferably in an amount of about 34% to about 70% by weight of the composition. Alternatively, when the surface active agent is a polyoxyethylene sorbitan fatty acid ester, the surface active agent may be present in an amount of about 40 mg to 185 mg per dose unit, and preferably, in an amount of about 85 mg per dose unit. The surface active agent may include both sodium ducosate and polyoxyethylene sorbitan fatty acid esters, wherein the sodium ducosate is present in an amount of about 17% to about 27% by weight of the composition and the polyoxyethylene sorbitan fatty acid esters is present in an amount of about 33% to about 57% by weight of the composition. When the surface active agent is sodium lauryl sulfate, the surface active agent may be present in an amount of about 10% to about 60% by weight of the composition. Alternatively, the surface active agent may be present in an amount of about 10 mg to 100 mg per dose unit, and preferably, in an amount of about 30 mg per dose unit.

Optionally, the composition may further comprise at least one pharmaceutically acceptable antioxidant. Antioxidants include, but are not limited to, vitamin E acetate, α-tocopherol, ascorbyl palmitate, butylated hydroxyanisole (BHA), butylated hydroxytoluene (BHT), propyl gallate, or tocopherol polyethyleneglycol succinate (TPGS).

The antioxidants are used in amounts known in the art to be effective and acceptable for pharmaceutical applications. When the antioxidant is vitamin E acetate, the vitamin E acetate is present in about 0.1% to 2% by weight of the formulation. Preferably, vitamin E acetate is present in about of 0.5 to 2 mg per dose and more preferably in about 1.4 mg per dose. When the antioxidant is α-tocopherol, the α-tocopherol is present in an amount of about 0.2% to 7% by weight of the formulation. Preferably, the α-tocopherol is present in an amount of about 2 to 7 mg per dose and more preferably in about 5 mg per dose. When the antioxidant is ascorbyl palmitate, ascorbyl palmitate is present in an amount of about 0.3% to 15% by weight of the formulation. Preferably, ascorbyl palmitate is present in an amount of about 5 to 15 mg per dose and more preferably in about 12 mg per dose. When the antioxidant is butylated hydroxyanisole (BHA), the BHA is present in an amount of about 0.2% to 7% by weight of the formulation. Preferably, the BHA is present in an amount of about 2 to 7 mg per dose and more preferably in about 5 mg per dose. When the antioxidant is butylated hydroxytoluene (BHT), the BHT is present in an amount of about 0.02% to 1% by weight of the formulation. Preferably, the BHT is present in an amount of about 0.2 to 1.0 mg per dose and more preferably in about 0.4 mg per dose. When the antioxidant is propyl gallate, the propyl gallate is present in an amount of about 0.002% to 0.2% by weight of the formulation. Preferably, the propyl gallate is present in an amount of 0.01 to 0.1 mg per dose and more preferably in about 0.05 mg per dose. When the antioxidant is tocopherol polyethyleneglycol succinate (TPGS), the TPGS is present in an amount of about 0.1% to 3% by weight ofthe formulation. Preferably, the TPGS is present in an amount of about 0.5 to 3.0 mg per dose and more preferably in about 2 mg per dose. Preferably, the antioxidants are vitamin E acetate or ascorbyl palmitate.

Optionally, the composition may further comprise a solid carrier. Solid carriers include, but are not limited to, at least one of microcrystalline cellulose, lactose, starch, sucrose, calcium phosphate, or sorbitol. Sucrose may be in pellet form, the starch in microcrystalline form, and the cellulose in powdered form. When present, the solid carriers are present in an amount of about 100 mg to about 400 mg per dose.

In one embodiment of the invention, the composition comprises a therapeutically effective amount of simvastatin or other statin drug dissolved in menthol and at least one surface active agent containing a sulfate moiety such as sodium lauryl sulfate or sodium ducosate. Optionally, at least one other surface active agent may be present in the composition. In another embodiment of the invention, the composition comprises a therapeutically effective amount of simvastatin in the closed lactone form dissolved in menthol, at least one surface active agent containing the sulfate moiety such as sodium lauryl sulfate or sodium ducosate and at least one second surface active agent, wherein at least 30% by weight of the dissolved simvastatin closed lactone form is hydrolyzed to the simvastatin open hydroxyl acid form when the composition is dissolved in 0.1N HCl. Optionally, the compositions of the invention may be absorbed onto a solid carrier. The therapeutically effective amount should be sufficient to treat high cholesterol, multiple sclerosis, and/or Alzheimer's disease.

In another embodiment of the invention, the composition comprises about 1% to 50% by weight simvastatin in the closed lactone form, about 10% to about 75% by weight menthol, about 25% to about 60% by weight polyoxyethylene sorbitan fatty acid esters or polyoxyethylene/polyoxypropylene copolymer, preferably polyoxyethylene sorbitan fatty acid esters, and about 10% to about 30% by weight of the composition sodium lauryl sulfate. In yet another embodiment of the invention, the composition comprises about 1% to 50% by weight simvastatin in the closed lactone form, about 15% to about 75% by weight menthol, about 25% to about 60% by weight polyoxyethylene sorbitan fatty acid esters or polyoxyethylene/polyoxypropylene copolymer, preferably, polyoxyethylene sorbitan fatty acid esters, and about 10% to about 30% by weight of the composition sodium ducosate. In yet another embodiment of the invention, the composition comprises about 8% by weight simvastatin in the closed lactone form, about 41% by weight menthol, about 34% by weight polyoxyethylene sorbitan fatty acid esters, or polyoxyethylene/polyoxypropylene copolymer, preferably polyoxyethylene sorbitan fatty acid esters, and about 17% by weight of the composition sodium ducosate. Although the present invention has been illustrated by the preceding embodiments, it will be apparent to those skilled in the art that many modifications, both to materials and amounts, may be practiced without departing from the scope of the invention.

The present invention also encompasses methods of preparing statin containing compositions. Generally, the pharmaceutically acceptable solvent and at least one surface active agent are mixed to form a first mixture. If necessary, the pharmaceutically acceptable solvent can be melted prior to adding other ingredients. Thereafter, the statin is added to the mixture and a solid or gel is formed, wherein the solid or gel is placed in capsules. The method may further comprise adding a solid carrier to the solid or gel, which subsequently is formed into a powder and the powder is placed in capsules or formed into tablets. Optionally, the method comprises adding to the melt a pharmaceutically acceptable antioxidant which is dissolved or dispersed in the melt.

In one embodiment, the method comprises: a) heating menthol to a temperature of about 40° C. to about 60° C. to effect melting thereof; b) adding at least one surface active agent to the menthol to form a first mixture; c) adding at least one second surface active agent containing a sulfate moiety to form a second mixture; d) stirring the second mixture until all the components have dissolved; e) adjusting the temperature of the second mixture to a temperature of about 50° C. to form a melt; f) adding simvastatin and stirring the simvastatin containing melt; and g) cooling the simvastatin containing melt to room temperature and dispensing the simvastatin containing melt into capsules, or alternatively, h) dispensing the simvastatin containing into capsules and cooling the simvastatin containing melt to room temperature. Alternatively, the method may further comprise adding at least one solid carrier such as microcrystalline cellulose, lactose, starch, sucrose, calcium phosphate, or sorbitol to the simvastatin containing melt, mixing the carrier into the simvastatin containing melt to form a third mixture, cooling the third mixture to room temperature to form a powder, and using the powder to produce pharmaceutical dosage forms such as capsules, tablets or sachets. Preferably, the surface active agent containing a sulfate moiety includes, but is not limited to, at least one of sodium ducosate or sodium lauryl sulfate.

In yet another embodiment, the invention encompasses methods of forming a solid simvastatin containing product comprising melting menthol at a temperature of about 40° C. to about 60° C. and adding simvastatin to the melt; stirring the melt until all the simvastatin dissolves; and adjusting the temperature of the melt to about 40° C. Optionally, the method comprises adding to the melt a pharmaceutically acceptable antioxidant which is dissolved or dispersed in the melt. In one embodiment, the melt composition comprises 50% to 90% by weight menthol, 8% to 50% by weight simvastatin, and 0% to 25% by weight antioxidant. Preferably, the melt composition comprises about 75% to 85% by weight of menthol, 10% to 25% by weight simvastatin, and 0% to 22% by weight antioxidant. In a more preferred embodiment, the melt composition comprises about 78% menthol and about 22% by weight simvastatin, if no antioxidant is present. If the antioxidant is present, then the composition comprises about 75% menthol, about 22% simvastatin, and about 3% vitamin E acetate. Alternatively, the composition comprises about 61% menthol, about 17% simvastatin, and about 21% ascorbyl palmitate by weight.

Prior to addition, a surface active agent granulate may be prepared in a separate vessel. For example, at least one surface active agent is melted and/or dissolved in a pharmaceutically acceptable solvent. Optionally, at least one pharmaceutically acceptable antioxidant may be added and dissolved. Thereafter, the melt or solution of the surface active agent is granulated with a solid carrier with and a pharmaceutically acceptable solvent and the solid granulate is dried and optionally milled. In one embodiment, Tween 80 is granulated with sorbitol USP powder, microcrystalline cellulose powder, or a combination thereof using ethanol (95%) or ethanol-water mixtures. In a preferred embodiment, about 15% to 40% by weight of Tween 80 is granulated with about 60% to 85% by weight sorbitol, microcrystalline cellulose, or a combination thereof. In a more preferred embodiment, about 10% to 40% by weight Tween 80, about 1% to 10% by weight of sodium ducosate, about 35% to 45% by weight of sorbitol, and about 30% to 40% by weight of microcrystalline cellulose are granulated using ethanol (95%) or ethanol-water mixtures. In a yet more preferred embodiment, about 15% to 30% by weight Tween 80, most preferably about 22% by weight, about 2% to 5% by weight of sodium ducosate, most preferably about 3.5% by weight, and about 35% to 45% by weight of sorbitol, most preferably about 39% by weight, and about 30% to 40% by weight of microcrystalline cellulose, most preferably about 36% by weight are granulated using a 1:1 ethanol-water mixture.

A preferred method of forming the granulation is by mixing the Tween 80 with about ⅕ volume ethanol (95%), adding the sodium ducosate and stirring with heating at 60° C. until the ducosate has dissolved. Adding the sorbitol and microcrystalline cellulose and granulating the mixture with about 15% w/w of 50% ethanol-water. Thereafter, the granulate is dried, e.g. in a fluid bed drier. The dry surface active agent granulate and the menthol-simvastatin melt are then mixed together and allowed to cool to room temperature with stirring. In a preferred embodiment about 5-10 parts of the granulate are mixed with one part of the simvastatin melt. In a more preferred embodiment about 7 parts of the granulate are mixed with 1 part of the simvastatin melt. The resulting composition may be used to further produce a solid dosage form or used as is.

The present invention also encompasses methods of treating a patient for elevated cholesterol levels, multiple sclerosis, and/or Alzheimer's disease comprising administering to a patient in need of such treatment a statin composition comprising a therapeutically effective amount of statin, at least one pharmaceutically acceptable solvent, and at least one surface active agent. The composition may remain in the patient's stomach under acidic aqueous conditions for a sufficient amount of time to allow for the formation of the open hydroxy acid form statin. Typically, a sufficient amount of time the composition remains in the stomach is from about 30 minutes to about 2 hours.

In one embodiment, the method of the invention further comprises a second surface active agent, wherein the second surface active agent contains a sulfate moiety such as sodium lauryl sulfate or sodium ducosate. In another embodiment, at least 30% the dissolved closed lactone form statin is hydrolyzed to the open hydroxy acid form statin when the composition is dissolved in 0.1N HCl. Preferably, the statin is simvastatin.

One embodiment of the method of treatment of the present invention encompasses treating a patient for elevated cholesterol levels comprising administering to a patient in need of such treatment a statin composition comprising a therapeutically effective amount of statin in the lactone form, menthol, and at least one surface active agent. Optionally, the composition comprises at least one pharmaceutically acceptable antioxidant. In one embodiment, the surface active agent contains a sulfate moiety such as sodium lauryl sulfate or sodium ducosate. In another embodiment, at least 30% the dissolved closed lactone form statin is hydrolyzed to the open hydroxy acid form statin when the composition is dissolved in 0.1N HCl.

In another embodiment, the present invention encompasses methods of treating a patient with multiple sclerosis comprising administering to a patient in need of such treatment a composition comprising a therapeutically effective amount of simvastatin in the lactone form dissolved in menthol, and at least one surface active agent. Optionally, the composition comprises at least one pharmaceutically acceptable antioxidant. Optionally, the surface active agent may contain a sulfate moiety, e.g. sodium lauryl sulfate or sodium ducosate. In one embodiment, at least 30% the simvastatin lactone is hydrolyzed to the simvastatin open hydroxyl acid form when the composition is dissolved in 0.1N HCl.

In another embodiment, the present invention encompasses methods of treating a patient with Alzheimer's disease comprising administering to a patient in need of such treatment a composition comprising a therapeutically effective amount of simvastatin in the lactone form dissolved in menthol, and at least one surface active agent. Optionally, the composition comprises at least one pharmaceutically acceptable antioxidant. Optionally, the surface active agent may contain a sulfate moiety, e.g. sodium lauryl sulfate or sodium ducosate. In one embodiment, at least 30% the simvastatin lactone is hydrolyzed to the simvastatin open hydroxyl acid form when the composition is dissolved in 0.1N HCl.

Typically, the statin is present in an amount sufficient for the treatment or prevention of high cholesterol or multiple sclerosis. In one embodiment, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease or disorder.

In certain embodiments, the compositions of the invention are administered to a patient, preferably a human, as a preventative measure against such diseases. As used herein, “prevention” or “preventing” refers to a reduction of the risk of acquiring a given disease or disorder. In a preferred mode of the embodiment, the compositions of the present invention are administered as a preventative measure to a patient, preferably a human having a genetic predisposition to a cardiovascular disease, high cholesterol, or multiple sclerosis.

As used herein, the term “high cholesterol” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org and http://rover.nhlbi.nih.gov/chd/, respectively). At the present time, the recommended level of IHDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Tables 1 and 2 illustrate the pre-systemic formation of simvastatin open hydroxy acid form from the simvastatin closed lactone form in 0.1N HCl using compositions of the present invention. The compositions should reside in the stomach under acidic aqueous conditions for about 30 minutes to about 2 hours. The in vitro resident times shown exemplify the amount of time necessary to allow for the preformation of the statin active metabolite, i.e. open hydroxy acid form. The pre-formed active metabolite could be absorbed in the small intestine adding to the amount of the active metabolite formed in the liver, thus potentially offering significant improvements to treatment with statins, preferably simvastatin.

The efficacy of the treatment as determined by the amount of statin open hydroxyl acid form in the system has determined the current treatment to be a superior treatment to that of prior art formulations such that formulations of the invention may be categorized as “superstatins.” The most serious, albeit rare, simvastatin side effect is muscular myopathy. The side effect is associated with the amount of closed lactone form simvastatin in the blood. Therefore, the compositions of the invention should not have any more side effects than the reference simvastatin.

Having described the invention with reference to certain preferred embodiments, other embodiments will become apparent to one skilled in the art from consideration of the specification. The invention is further defined by reference to the following examples describing in detail the compositions, preparation of the compositions, and methods of administration of the invention. It will be apparent to those skilled in the art that many modifications, both to materials and methods, may be practiced without departing from the scope of the invention.

### EXAMPLES

### Example 1

### In vitro Hydrolysis of Simvastatin Formulations under Simulated Gastric Conditions

Generally, the simvastatin formulations were prepared by heating menthol to about 60° C. while stirring and adding a surface active agent (either TWEEN® or Poloxamer) and sodium ducosate or sodium lauryl sulfate. The mixture was stirred until all the components dissolved to form a melt. Thereafter, the melt was cooled to about 50° C., simvastatin was added during stirring until dissolved, the mixture was cooled to room temperature, and dispensed into capsules. After about an hour the liquid in the capsule had gelled. Each capsule was filled with enough formulation to have a dose of 20 mg of simvastatin.

Each formulation was tested by dissolution in 50 ml of 0.1N HCl at 37° C. with stirring. The amount of dissolved simvastatin lactone and simvastatin hydroxyl acid were determined by HPLC on a column (ODS BDS 150×4.6 mm, 5 micron), mobile phase: 40:60 dilute phosphoric acid:acetonitrile at a flow rate of 2 ml/min and detected with a UV wavelength of 238 nm. It was determined that the retention times for the simvastatin hydroxyl acid was 3.6 minutes and simvastatin lactone was 5.7 minutes.

The dissolution results for each formulation are summarized in Tables 1 and 2.

TABLE 1
Simvastatin Formulation^{a}

| | | TWEEN ® | TWEEN ® | Poloxamer | Sodium | | % Dissolved 60 | % converted to | % hydrolyzed |
|---|---|---|---|---|---|---|---|---|---|
| Simvastatin | Menthol | 80 | 20 | 407 | Ducosate | SLS | min 0.1 N HCl^{b} | hydroxyl acid^{c} | Simvastatin^{d} |
| 100 | 0 | 0 | 0 | 0 | 0 | 0 | 0 | 0 | 0 |
| 16 | 84 | 0 | 0 | 0 | 0 | 0 | 11 | 19 | 2 |
| 5 | 27 | 0 | 0 | 68 | 0 | 0 | 97 | 23 | 22 |
| 5 | 27 | 68 | 0 | 0 | 0 | 0 | 97 | 29 | 28 |
| 8 | 40 | 52 | 0 | 0 | 0 | 0 | 96 | 25 | 24 |
| 7 | 50 | 43 | 0 | 0 | 0 | 0 | 100 | 19 | 19 |
| 7 | 50 | 22 | 0 | 21 | 0 | 0 | 88 | 15 | 13 |
| 8 | 40 | 0 | 52 | 0 | 0 | 0 | 95 | 16 | 15 |
| 4 | 40 | 0 | 0 | 0 | 0 | 55 | 98 | 41 | 40 |
| 5 | 27 | 0 | 0 | 0 | 68 | 0 | 21 | 41 | 9 |
| 10 | 49 | 21 | 0 | 0 | 21 | 0 | 67 | 42 | 28 |
| 9 | 44 | 38 | 0 | 0 | 9 | 0 | 88 | 41 | 36 |
| 8 | 41 | 34 | 0 | 0 | 17 | 0 | 90 | 46 | 41 |

^{a}All ingredients amounts are given in % weight of the composition.
^{b}Total amount of dissolved simvastatin (closed lactone form and open hydroxy acid form).
^{c}Weight % of the total dissolved simvastatin that is in the open hydroxy acid form.
^{d}Weight % of the total amount of hydrolyzed Simvastatin from the initial Simvastatin amount.

Table 1 illustrates that simvastatin lactone alone does not dissolve well in the water (0% dissolved) and has no conversion under these conditions to the hydroxyl acid (0%). The dissolution of simvastatin lactone formulations and menthol ranged from a low value of 11% with almost 20% of this material hydrolyzing to the acid. The addition of various surfactants, such as TWEEN® 80, TWEEN® 20, or Poloxamer 407, allowed for about total dissolution of simvastatin, wherein about 20% to about 30% of the lactone had converted to the hydroxyl acid form. When sodium lauryl sulfate (SLS) was the surface active agent, the amount of conversion of the lactone form to acid increased to above 40%. Sodium ducosate as the only surface active agent yielded a conversion of over 40%, however, the simvastatin dissolution in the acidic water was low (21%). Mixtures of TWEEN® 80 and sodium ducosate gave formulations that were both highly soluble (90%) within one hour with conversions of the lactone to the hydroxyl acid of over 40%.

TABLE 2
Simvastatin Formulations by weight and percent of components

| | | | | | | | | % Dissolved | | |
|---|---|---|---|---|---|---|---|---|---|---|
| | | | | TWEEN ® | | Sodium | | 30 min 0.1 N | % converted to | % hydrolyzed |
| Simvastatin | | Menthol | | 80 | | Ducosate | | HCl^{a} | hydroxyl acid^{b} | Simvastatin^{c} |
| mg | % | mg | % | mg | % | mg | % | % | % | % |
| 20 | 8.1 | 100 | 40.5 | 85 | 34.4 | 42 | 17.0 | 83 | 42 | 35 |
| 20 | 9.0 | 75 | 33.8 | 85 | 38.3 | 42 | 18.9 | 68 | 42 | 29 |
| 20 | 10.2 | 50 | 25.4 | 85 | 43.1 | 42 | 21.3 | 77 | 41 | 32 |
| 20 | 11.6 | 25 | 14.5 | 85 | 49.4 | 42 | 24.4 | 72 | 41 | 30 |
| 20 | 12.5 | 12.5 | 7.8 | 85 | 53.3 | 42 | 26.3 | 72 | 43 | 31 |
| 20 | 13.2 | 5 | 3.3 | 85 | 55.9 | 42 | 27.6 | 8 | 47 | 4 |

^{a}Total amount of dissolved simvastatin (closed lactone form and open hydroxy acid form).
^{b}Weight % of the total dissolved simvastatin that is in the open hydroxy acid form.
^{c}Weight % of the total amount of hydrolyzed Simvastatin from the initial Simvastatin amount.

Table 2 illustrates the effect of lowering the amount of menthol in the simvastatin formulation from 40% to 3% by weight while keeping all the other ingredient amounts constant. Menthol is effective at about 8% w/w but at 3% w/w simvastatin solubility diminishes.

### Example 2

### Solid Simvastatin-Menthol Formulation

Into a jacketed glass reactor were added 100 grams of Tween 80, 11.7 grams of sodium ducosate, and 20 grams of ethanol (95%). The mixture was stirred and heated to 60° C. for about an hour or until all the sodium ducosate had dissolved. Under continuous stirring by a laboratory mechanical stirrer, 176 grams of sorbitol (USP) powder were slowly added followed by 161 grams of microcrystalline cellulose (Avicel PH102™, FMC International) to form a wet mass. The wet mass was transferred to a Diosna P⅙ high shear granulator. About 70 grams of 50% ethanol-water were added to the granulator and the mass was granulated at a speed of 380 rpm for 5 minutes. The wet granulate was dried in a Diosna Mini Lab fluidized bed drier to less than 2% volatiles at an inlet temperature of 50° C. and a fan set point of 40%. The volatile content was tested at 105° C. using a Sartorious MA 30 LOD tester. The yield of dry granulate was 382 grams (85%).

In a separate jacketed reactor heated to 60° C. were mixed 10 grams of simvastatin (micronized, Biogal Inc.) and 35 grams of menthol (USP). The homogeneous melt was cooled to 40° C. and stirred at about 100 rpm. Vitamin E acetate (BASF) (1.4 grams) was added and dispersed. The dry granulate (382 grams) was added to the melt under constant stirring over a period of 5 minutes. The stirring was continued another 15 minutes after which the formed powder was removed and allowed to cool for three hours. Capsules were filled with 428 mg of the powder for a 10 mg simvastatin dose per capsule.

The formulation was tested for dissolution as described in Example 1. After 30 minutes, 69.4%±4.6% had dissolved with 42% being in the form of simvastatin hydroxyl acid.

### Example 3

### PK Study of Simvastatin Formulations

To determine the bioavailability of simvastatin, a randomized, 4-way crossover comparative bioavailability study with three simvastatin test formulations (20 mg, A, B, C) as compared to a simvastatin (20 mg, D) reference was performed with healthy male volunteers. The pharmacokinetic profiles (Cₘₐₓ, Tₘₐₓ, and AUC) of simvastastin hydroxy acid and simvastatin lactone-were observed following administration of single doses of several simvastatin/menthol test formulations as compared to the administration of the simvastatin reference (no menthol), in twelve fasting healthy male volunteers.

Both the test (A, B, and C) and reference (D) drug formulations were administered as a single oral dose, with 240 ml of water following an overnight fast of at least 10 hours. Subjects were monitored to ensure that the tablet and capsules are swallowed whole and not chewed.

Each of the simvastatin/menthol test formulations was supplied as a 20 mg simvastatin+100 mg menthol capsule, with each test formulation provided as a clearly distinguishable capsule of different color. All subjects received the test and reference drug formulations in a four-way crossover design. Subjects were randomly assigned to the Reference formulation or to one of the three Test formulations (D; A; B; C), and were crossed over the following week to an alternative treatment. The cycle was repeated for 2 more study sessions, so that all subjects had received all 4 study treatments in a unique, subject-specific order, with a 1 week wash-out period between treatments. All subjects, regardless of the order in which they received their treatment assignment, received a total exposure of 80 mg simvastatin and 300 mg menthol over the 4 study sessions. Twelve (12) healthy adult male volunteers, 18-55 years of age participated in the study.

The subjects provided a “0” hour blood sample within 1-1.5 hours prior to initial dosing. Thereafter, each subject was dosed with the study drug (Reference 1, Test 1, Test 2, or Test 3) in the fasted state, according to the individual randomization scheme for each subject. All subjects, regardless of treatment assignment, were monitored in the clinic for the first 12 hours following initial dosing, with serial blood samples taken periodically over the first 12 hours following dosing. Subjects were discharged from the clinic after 12 hours and be asked to return to the clinic a week later for the next study session. A total of 11 blood samples were collected from each subject at each study session.

From each patient blood samples (10 ml) were obtained from all subjects regardless of treatment assignment at the following time points: “0” hour (pre-dosing), 0.5, 1, 1.5, 2, 3, 4, 6, 8, 10 and 12 hours post-initial dosing, for a total of 11 blood samples per study period (110 ml total). The blood was placed in EDTA test tubes, centrifuged at 3000 rpm for 10 minutes at 4° C., with the plasma separated for subsequent analysis of simvastatin lactone and simvastatin hydroxy acid, each was sent to the analytical laboratory for analysis using a validated LC/MS/MS method, with a calibration range of 0.3-28 ng/ml.

The study was repeated after at least a 7 day wash-out period between dosings, with each subject crossed over to the alternative treatment arm as per their unique randomization code, and followed as described below for a total of 4 study sessions.

TABLE 3
Description of Treatments

| Treatment | Description |
|---|---|
| A | 1 Capsule containing 20 mg simvastatin |
| | dissolved in 100 mg menthol |
| B | 1 Capsule containing 20 mg simvastatin |
| | dissolved in 100 mg menthol and 85 mg |
| | TWEEN ® 80 and 42 mg sodium ducosate |
| C | 2 capsules each containing 10 mg sim- |
| | vastatin dissolved in 42 mg menthol and |
| | 42 mg TWEEN ® 80 and 21 mg sodium |
| | ducosate loaded on 100 mg of micro- |
| | crystalline cellulose (AVICEL ® PH102) |
| D (Reference) | 1 Tablet containing 20 mg simvastatin |
| | previously shown to be bioequivalent |
| | to Zocor ® |

The four treatment groups are described in Table 3. A summary of the pharmacokinetic results of the AUC (reflecting the amount of the drug in the blood) and Cₘₐₓ (maximum concentration of drug in the blood) data are shown in Table 4 for the simvastatin lactone and in Table 5 for the simvastatin hydroxyl acid. Treatments B and D (reference) yield similar results in AUC for the simvastatin lactone (ratio B/D=0.97). However, the ratio of active metabolite simvastatin hydroxyl acid is greater than five (ratio B/D=5.1). The Cₘₐₓ data also illustrates the higher concentration of acid to lactone as compared to the reference sample D, B/D=1.97 for the simvastatin lactone and 5.0 for the hydroxyl acid. As simvastatin efficacy is dependent upon the availability of the hydroxyl acid, treatment B was determined to be vastly superior to the reference sample. For treatment B, the value for the side effect of muscular myopathy is unchanged as compared to the reference sample.

TABLE 4
AUC and Cₘₐₓ for simvastatin lactone

| Treatment | A | B | C | D | A/D | B/D | C/D |
|---|---|---|---|---|---|---|---|
| Mean AUC ng*h/ml | 20.2 ± 16.8 | 11.7 ± 6.8 | 3.9 ± 2.3 | 12.1 ± 7.1 | 1.67 | 0.97 | 0.32 |
| Mean Cₘₐₓ ng/ml | 8.1 ± 5.3 | 6.3 ± 6.4 | 2.2 ± 1.2 | 3.3 ± 2.0 | 2.45 | 1.91 | 0.67 |

TABLE 5
AUC and Cₘₐₓ for simvastatin hydroxyl acid

| Treatment | A | B | C | D | A/D | B/D | C/D |
|---|---|---|---|---|---|---|---|
| Mean AUC ng*h/ml | 11.8 ± 4.8 | 38.3 ± 20.8 | 20.2 ± 11.4 | 7.5 ± 3.7 | 1.57 | 5.1 | 2.69 |
| Mean Cₘₐₓ ng/ml | 1.4 ± 0.6 | 5.5 ± 3.2 | 3.1 ± 2.2 | 1.1 ± 0.6 | 1.27 | 5 | 2.81 |

In treatment C the simvastatin/menthol/surfactant formulation was adsorbed onto a solid carrier. The AUC for the simvastatin hydroxyl acid for treatment C is 269% compared to reference sample D, thus, for the reasons discussed in treatment B, treatment C is expected to be more efficacious than the reference D. The ratio (C/D) for the unchanged lactone is 0.32 indicating that treatment C provides less of the unchanged lactone than the reference sample D. Therefore, treatment C may improve treatment for lowering cholesterol and have considerably less side effects.

Treatment A, wherein simvastatin was dissolved in menthol without surfactants, gave ratio of 1.67 (A/D) and 1.57 for the AUC's of the unchanged lactone and hydroxyl acid forms, respectively. This result illustrates approximately 50% to 60% higher absorption of simvastatin and a similar rise in the presence of the active metabolite.

1. A composition comprising:
a statin;
at least one pharmaceutically acceptable solvent; and
at least one surface active agent,
wherein about 9% to about 50% by weight of the statin is hydrolyzed from a closed lactone form to an open hydroxy acid form when the composition is placed in an aqueous acidic solution.

2. The composition according to claim 1, wherein the aqueous acidic solution is 0.1N HCl.

3. The composition according to claim 1, wherein the statin is lovastatin, simvastatin, or a combination thereof.

4. The composition according to claim 1, wherein the statin is simvastatin.

5. The composition according to claim 1, wherein at least 90% by weight of the total amount of statin is present in a closed lactone form statin.

6. The composition according to claim 1, wherein the statin is present in an amount of about 1% to about 50% by weight of the composition.

7. The composition according to claim 1, wherein the pharmaceutically acceptable solvent is ethanol, propylene glycol, glycerol, isopropanol, butanol, or menthol.

8. The composition according to claim 1, wherein the pharmaceutically acceptable solvent is menthol.

9. The composition according to claim 1, wherein the pharmaceutically acceptable solvent is present in an amount of about 10% to about 75% by weight of the composition.

10. The composition according to claim 1, wherein the surface active agent is glyceryl ester, polyoxyethylene glycol ester, polyoxyethylene glycol ether, polyoxyethylene sorbitan fatty acid ester, polyoxyethylene/polyoxypropylene copolymer, sodium lauryl sulfate, or sodium ducosate.

11. The composition according to claim 1, wherein the surface active agent is present in an amount of about 5% to about 85% by weight of the composition.

12. The composition according to claim 1, further comprising at least one antioxidant.

13. The composition according to claim 12, wherein the antioxidant is vitamin E acetate, α-tocopherol, ascorbyl palmitate, butylated hydroxyanisole, butylated hydroxytoluene, propyl gallate, or tocopherol polyethyleneglycol succinate.

14. The composition according to claim 12, wherein the antioxidant is vitamin E acetate or ascorbyl palmitate.

15. The composition according to claim 13 wherein the vitamin E acetate is present in an amount of about 0.1% to about 2% by weight of the composition.

16. The composition according to claim 13 wherein the ascorbyl palmitate is present in an amount of about 0.3% to about 15% by weight of the composition.

17. The composition according to claim 1, comprising at least two surface active agents wherein the first surface active agent is polyoxyethylene sorbitan fatty acid ester or polyoxyethylene/polyoxypropylene copolymer and the second surface active agent is sodium lauryl sulfate or sodium ducosate.

18. The composition according to claim 15, wherein the first surface active agent is polyoxyethylene sorbitan fatty acid ester and the second surface active agent is sodium ducosate.

19. The composition according to claim 1, wherein when the composition is dissolved in an aqueous acidic solution, at least 20% by weight of the dissolved statin is hydrolyzed from a closed lactone form to an open hydroxy acid form.

20. The composition according to claim 1 in a pharmaceutically acceptable dosage form.

21. A composition comprising:
simvastatin;
menthol; and
polyoxyethylene sorbitan fatty acid ester,
wherein about 9% to about 50% by weight of the simvastatin is hydrolyzed from a closed lactone form to an open hydroxy acid form when the composition is placed in an aqueous acidic solution.

22. The composition according to claim 21, wherein the aqueous acidic solution is 0.1N HCl.

23. The composition according to claim 21, wherein the simvastatin is present in an amount of about 1% to 50% by weight of the composition and menthol is present in an amount of about 10% to about 75% by weight of the composition.

24. The composition according to claim 21, comprising at least two surface active agents wherein the first surface active agent is polyoxyethylene sorbitan fatty acid ester or polyoxyethylene/polyoxypropylene copolymer and the second surface active agent is sodium lauryl sulfate or sodium ducosate.

25. The composition according to claim 24, wherein the polyoxyethylene sorbitan fatty acid ester is present in an amount of 33% to about 57% by weight and the sodium lauryl sulfate or sodium ducosate is present in an amount of about 5% to about 70% by weight.

26. The composition according to claim 21, wherein when the composition is dissolved in an aqueous acidic solution, at least 20% by weight of the dissolved simvastatin is hydrolyzed from a closed lactone form to an open hydroxy acid form.

27. The composition according to claim 21, further comprising at least one antioxidant.

28. The composition according to claim 21, wherein the antioxidant is vitamin E acetate, α-tocopherol, ascorbyl palmitate, butylated hydroxyanisole, butylated hydroxytoluene, propyl gallate, or tocopherol polyethyleneglycol succinate.

29. The composition according to claim 21, wherein the antioxidant is vitamin E acetate or ascorbyl palmitate.

30. The composition according to claim 29, wherein the vitamin E acetate is present in an amount of about 0.1% to about 2% by weight of the composition.

31. The composition according to claim 29, wherein the ascorbyl palmitate is present in an amount of about 0.3% to about 15% by weight of the composition.

32. A method of preparing a simvastatin containing composition comprising:
(a) heating menthol to a temperature of about 40° C. to about 60° C. to effect melting thereof;
(b) adding at least one first surface active agent to the menthol to form a first mixture;
(c) adding at least one second surface active agent containing a sulfate moiety and stirring until all components have dissolved to form a second mixture;
(d) adjusting the temperature of the second mixture to a temperature of about 50° C. to form a melt;
(e) adding simvastatin to the melt to form a simvastatin containing melt; and
(f) cooling the simvastatin containing melt to room temperature and dispensing the simvastatin containing melt into capsules or alternatively dispensing the simvastatin melt into capsules and cooling the simvastatin melt to room temperature.

33. The method according to claim 32, further comprising adding at least one solid carrier to the cooled simvastatin containing melt to form a third mixture prior to dispensing the simvastatin containing melt into capsules.

34. The method according to claim 33, wherein the solid carrier is microcrystalline cellulose, lactose, or sorbitol.

35. The method according to claim 33, further comprising cooling the third mixture to room temperature to form a powder.

36. The method according to claim 32, further comprising adding at least one pharmaceutically acceptable antioxidant when the first surface active agent is added to menthol.

37. The method according to claim 36, wherein the antioxidant is vitamin E acetate, α-tocopherol, ascorbyl palmitate, butylated hydroxyanisole, butylated hydroxytoluene, propyl gallate, or tocopherol polyethyleneglycol succinate.

38. The method according to claim 36, wherein the antioxidant is vitamin E acetate or ascorbyl palmitate.

39. A method of preparing a simvastatin containing composition comprising:
(a) heating menthol to a temperature of about 40° C. to about 60° C. to effect melting thereof;
(b) adding simvastatin to the melt and effecting dissolution thereof;
(c) adjusting the temperature of the melt to 40° C. while stirring to form a simvastatin menthol solution;
(d) melting or dissolving at least one surface active agent and at least one second surface active agent containing a sulfate moiety, to form a surface active agent mixture;
(e) combining at least one pharmaceutically acceptable solid carrier with the surface active agent mixture and granulating to form a surface active agent granulate;
(f) drying the surface active agent granulate;
(g) mixing the surface active agent granulate with the simvastatin menthol solution to form a simvastatin containing composition.

40. The method according to claim 39, wherein the solid carrier is microcrystalline cellulose, lactose, or sorbitol.

41. The method according to claim 39, further comprising adding at least one pharmaceutically acceptable antioxidant to the surface active agent mixture.

42. The method according to claim 41, wherein the antioxidant is vitamin E acetate, α-tocopherol, ascorbyl palmitate, butylated hydroxyanisole, butylated hydroxytoluene, propyl gallate, or tocopherol polyethyleneglycol succinate.

43. The method according to claim 41, wherein the antioxidant is vitamin E acetate or ascorbyl palmitate.

44. The method according to claim 41, wherein at least one pharmaceutically acceptable solvent is added to the first surface active agent and second surface active agent.

45. A method for treating a patient for elevated cholesterol levels, multiple sclerosis, or Alzheimer's disease comprising:
administering to a patient in need of such treatment a statin composition comprising a therapeutically effective amount of a statin, at least one pharmaceutically acceptable solvent, and at least one surface active agent, wherein about 9% to about 50% by weight of the statin is hydrolyzed from a closed lactone form to an open hydroxy acid form when the composition is placed in an aqueous acidic solution.

46. The method according to claim 45, wherein the aqueous acidic solution is 0.1 N HCl.

47. The method according to claim 45, wherein the statin is simvastatin.

48. The method according to claim 45, wherein the statin is simvastatin present in an amount of about 1% to 50% by weight of the composition and the pharmaceutically acceptable solvent is menthol present in an amount of about 10% to about 75% by weight of the composition.

49. The method according to claim 45, wherein the composition comprises at least two surface active agents wherein the first surface active agent is polyoxyethylene sorbitan fatty acid ester or polyoxyethylene/polyoxypropylene copolymer and the second surface active agent is sodium lauryl sulfate or sodium ducosate.

50. The method according to claim 49, wherein the polyoxyethylene sorbitan fatty acid ester is present in an amount of 33% to about 57% by weight and the sodium lauryl sulfate or sodium ducosate is present in an amount of about 5% to about 70% by weight.

51. The method according to claim 45, wherein the composition further comprises at least one antioxidant.

52. The method according to claim 45, wherein when the composition is dissolved in 0.1N HCl, at least 30% by weight of the dissolved statin is hydrolyzed from the closed lactone form to the open hydroxy acid form.

53. A method for treating a patient for elevated cholesterol levels comprising administering to a patient in need of such treatment a statin composition comprising a therapeutically effective amount of a statin, menthol, and at least one surface active agent,
wherein about 9% to about 50% by weight of the statin is hydrolyzed from a closed lactone form to an open hydroxy acid form when the composition is placed in an aqueous acidic solution.

54. The method according to claim 53, wherein the aqueous acidic solution is 0.1N HCl.

55. The method according to claim 53, wherein the statin is simvastatin.

56. The method according to claim 53, wherein the statin is simvastatin present in an amount of about 1% to 50% by weight of the composition and the menthol is present in an amount of about 10% to about 75% by weight of the composition.

57. The method according to claim 53, wherein the composition comprises at least two surface active agents wherein the first surface active agent is polyoxyethylene sorbitan fatty acid ester or polyoxyethylene/polyoxypropylene copolymer and the second surface active agent is sodium lauryl sulfate or sodium ducosate.

58. The method according to claim 57, wherein the polyoxyethylene sorbitan fatty acid ester is present in an amount of 33% to about 57% by weight and the sodium lauryl sulfate or sodium ducosate is present in an amount of about 5% to about 70% by weight.

59. The method according to claim 53, wherein when the composition is dissolved in 0.1N HCl, at least 30% by weight of the dissolved statin is hydrolyzed from the closed lactone form to the open hydroxy acid form.

60. A method for treating a patient with multiple sclerosis comprising administering to a patient in need of such treatment a composition comprising a therapeutically effective amount of simvastatin dissolved in menthol, and at least one surface active agent,
wherein about 9% to about 50% by weight of the simvastatin is hydrolyzed from a closed lactone form to an open hydroxy acid form when the composition is placed in an aqueous acidic solution.

61. The method according to claim 60, wherein the aqueous acidic solution is 0.1N HCl.

62. The method according to claim 60, wherein the simvastatin is present in an amount of about 1% to 50% by weight of the composition and the menthol is present in an amount of about 10% to about 75% by weight of the composition.

63. The method according to claim 60, wherein the composition comprises at least two surface active agents wherein the first surface active agent is polyoxyethylene sorbitan fatty acid ester or polyoxyethylene/polyoxypropylene copolymer and the second surface active agent is sodium lauryl sulfate or sodium ducosate.

64. The method according to claim 63, wherein the polyoxyethylene sorbitan fatty acid ester is present in an amount of 33% to about 57% by weight and the sodium lauryl sulfate or sodium ducosate is present in an amount of about 5% to about 70% by weight.

65. The method according to claim 60, wherein when the composition is dissolved in 0.1N HCl, at least 30% by weight of the dissolved simvastatin is hydrolyzed from the closed lactone form to the open hydroxy acid form.

66. The method according to claim 60, wherein the composition firther comprises at least one antioxidant.

67. A method for treating a patient with Alzheimer's disease comprising administering to a patient in need of such treatment a composition comprising a therapeutically effective amount of simvastatin dissolved in menthol, and at least one surface active agent,
wherein about 9% to about 50% by weight of the simvastatin is hydrolyzed from a closed lactone form to an open hydroxy acid form when the composition is placed in an aqueous acidic solution.

68. The method according to claim 67, wherein the surface active agent is polyoxyethylene sorbitan fatty acid ester, polyoxyethylene/polyoxypropylene copolymer, sodium lauryl sulfate, or sodium ducosate.
